(19) United States
(12) Patent Application Publication
(10) Pub. No.: US 2014/0274809 A1
(43) Pub. Date: Sep. 18, 2014

(54) MULTI-WELL MANIFOLD ASSEMBLY SYSTEM FOR OLIGONUCLEOTIDE SYNTHESIS

(71) Applicant: Integrated DNA Technologies, Inc., Skokie, IL (US)

(72) Inventors: Jon Harvey, South Amana, IA (US); Ryan Witt, Oxford, IA (US); William E. Martin, III, Coralville, IA (US)

(21) Appl. No.: 14/211,431

(22) Filed: Mar. 14, 2014

Related U.S. Application Data
(60) Provisional application No. 61/789,341, filed on Mar. 15, 2013.

(51) Int. Cl. B01L 3/00; C12N 15/10

(52) U.S. Cl. CPC B01L 3/502; C12N 15/1093
USPC 506/26; 506/40

(57) ABSTRACT

A multi-well manifold assembly and method for reducing cross-contamination in continuous synthesis reactions in channels of microfluidic devices, for example oligonucleotide synthesis.

### CROSS-REFERENCE TO RELATED APPLICATIONS

This patent application claims priority to U.S. Provisional Patent Application No. 61/789,341 filed Mar. 15, 2013, the content of which is incorporated herein by reference in its entirety.

### FIELD OF THE INVENTION

The present invention relates to an apparatus and methods for reducing cross-contamination in synthesis reactions, such as, for example, oligonucleotide synthesis reactions.

### BACKGROUND OF THE INVENTION

Synthetic DNA sequences are a vital tool in molecular biology. They are used in gene therapy, vaccines, DNA libraries, environmental engineering, diagnostics, tissue engineering and research into genetic variants. Currently there are a number of methods for oligonucleotide synthesis, although most methods use phosphoramidite chemistry. Oligonucleotide synthesis occurs in support columns, or in high throughput, multiwell plates having an array of wells. Multiwell plates provide the ability to carry out multiple reactions at one time and increase throughput. Generally, wells are formed via injection molding trays, or such wells can be machined out of a rigid section of material to form multiwall plates. They are commercially available through a number of vendors.

Oligonucleotide synthesis often occurs on a solid support such as controlled pore glass (CPG), or on thermoplastics such as polystyrene or polyethylene, or a combination thereof can be mixed or sintered together to form a membrane support. In whatever form, the supports are placed within the wells of the multiwell plate. In one embodiment the well has a top opening where the support and reagents enter the well, and a bottom opening where the reagent exits, but the support is otherwise retained in the well through its size or a further barrier such as a frit (for examples of multiwell plates, support membranes and frits, see U.S. Pat. No. 8,129,517, hereby incorporated by reference in its entirety). The supports are utilized as reversible attachment points for the initiation and growth of the custom oligonucleotide. The array of wells of a multiwell plate allow for simultaneous manufacturing of multiple custom oligonucleotide sequences. Selected reagents are added to individual wells in order to generate the desired oligonucleotide sequence. The process of adding the selected reagents to the appropriate well is repeated until the growing oligonucleotide strand is complete and the desired product is obtained. In addition modified nucleotides or chemical modifications can be attached sequentially to the growing oligonucleotide strand. Additional modifications can consist of, and are not limited to, dyes such as fluorophores or quenchers; labels or nucleotides modified with labels; and modifications such as modified bases and/or modified linkers. Once the desired oligonucleotide is created the oligonucleotide is released from the support structure.

Batch process trays are typically utilized to provide multiple wells having a bottom outlet in communication with a vacuum chamber. Liquid deposited in each well flows through the solid support and the percolate or eluate passes by way of the bottom outlets into the vacuum chamber serving as a waste reservoir. The reagents exit the wells through gravity, or the wells are subjected to a negative pressure differential such as a vacuum to increase the rate of through-flow for the procedure.

Deprotection of oligonucleotides is carried out after the synthesis of the oligonucleotide is complete, cleaving the DNA from the solid support and removing protecting groups through treatment with ammonia. Treatment with ammonia is typically carried out generally by way of two methods. In a first method, a liquid ammonia solution is used; in a second method ammonia gas is used. To cleave the oligonucleotide from the support matrix and completely remove protecting groups, wherein an aqueous ammonia solution is used, the support bound product is treated with concentrated ammonia.

Anhydrous gas-phase deprotection is frequently utilized within oligonucleotide synthesizers containing multi-well reaction plates, providing deprotection of oligonucleotides via parallel deprotection of multi-well assay columns. Because no water is present and the fully deprotected oligonucleotides remain adsorbed to the solid support, cross-contamination has been considered lower. However, with advanced quantitative measures in the pharmaceutical and biotech industry, and the necessary purity levels needed in oligonucleotide synthesis, even low levels of cross-contamination must be prevented. Current biological technologies can detect small quantities of cross-contamination that are unacceptable for further use. As a result, resultant oligonucleotides often require further purification.

A number of factors can contribute to gas phase cross-contamination. One factor is the temperature/pressure changes causing the ammonia gas to move between wells. Another observed factor is that material comes out like a fog, dripping or dispersing condensed material against the bottom of the plate so that it becomes wetted with a mixture of condensate from the wells. That material is believed to form a residual condensate mixture which falls into additional plates, creating a mixture which gets cross-contaminated into other wells.

Cross-contamination of the nucleic acid samples in multiwell plates poses significant challenges for multiwell synthesis reactions. Numerous techniques have been used in trying to prevent cross-contamination. For example, in one method a pressure differential is applied to the vial bottom opening. Bailey et al., U.S. Patent Publication US 2012/0085415.

Various attempts to reduce cross-contamination include or could include adjusting the engagement of the multiwall plate in the reaction vessel, adjusting nozzle design of the plate assembly, or making the wells conical shaped to taper at the bottom (see Cheng et al., Nuc. Acids Res., Sep. 15, 2002; 30(18): e93). Alternatively, a technician could resort to manual liquid transfer, avoiding differential pressure transfers under vacuum, i.e. via pipette. However, such liquid transfer processes are significantly more time consuming and costly.

Despite these attempts, cross-contamination still remains a significant issue in multiwell synthesis reaction systems. Thus, it is desired to develop additional techniques to reduce cross-contamination during multiwell oligonucleotide synthesis.

### SUMMARY OF THE INVENTION

The present invention is directed to a system and method for reducing cross-contamination in multiwell synthesis. The present invention substantially eliminates cross-contamination in synthesis reactions. More specifically, the present invention relates to a specific construction of a multi-well manifold assembly and use of positive pressure during the synthesis reactions for use in continuous flow reactions that reduces cross-contamination between wells. Reduction of cross-contamination improves the efficiency, quality and reproducibility of synthesis reactions.

In one embodiment the multi-well manifold assembly for the reduction of cross-contamination during synthesis comprises a full ring velocity stack plate having a full ring opening with a rim and a plurality of apertures for receiving a plurality of elongated tubes. The assembly further includes a tube manifold received within the full ring opening of the full ring velocity stack plate. The tube manifold is constructed having a main body and top periphery with a top surface. A plurality of channels extends through the top surface, top periphery and traverse through the main body of the tube manifold. Pluralities of inserts are received within the channels mounted within the tube manifold, the inserts aligning with and mating with the elongated tubes. At least one sealing means is provided for sealingly engaging the stack plate, tube manifold, inserts and elongated tubes. As constructed, the tube manifold is inserted and secured within the full ring opening of the full ring velocity stack plate. It tightly abuts the sealing means and the stack plate to substantially form a seal between the tube manifold and the stack plate to reduce cross-contamination of synthesis reactions in oligonucleotide synthesis instruments.

In one embodiment, the multi-well manifold assembly is used specifically for the deprotection step of oligonucleotide synthesis. In another embodiment, the multiwall manifold assembly is incorporated into a high throughput oligonucleotide synthesizer.

In one embodiment the inserts are gas phase inserts comprised of any material that is neutral to deprotection. In one embodiment, the gas phase inserts are made of PVC EPDM. If the manifold assembly is incorporated with a synthesizer, wherein additional steps or all steps of synthesis occur, the inserts are made of a material neutral to oligonucleotide synthesis, such as Teflon®.

In another contemplated embodiment, the sealing means is a velocity stack o-ring located on the rim of the opening of the full ring velocity stack plate. Preferably the sealing means is an o-ring located on an underside of the top periphery of the tube manifold. Most preferably, the sealing means includes at least two o-rings. In this embodiment, at least one sealing means includes a velocity stack o-ring located on the rim of the opening of the full ring velocity stack plate and wherein at least one sealing includes an o-ring located on an underside of the top periphery of the tube manifold.

Other embodiments concern securement of the tube manifold from the full ring velocity stack plate by attachment means. Preferably, attachment means includes screwing of the tube manifold to the full ring velocity stack plate, although a number of known attachment means may be utilized without departing from the scope of the invention. The assembly may include a plurality of assay plates for use with the system, or selling the assembly as a kit; alternatively, the assay plates may be sold separately.

In one embodiment, the top periphery of the tube manifold extends outwardly from the main body forming a shelf. Upon insertion within the opening of the full ring velocity stack plate the shelf or top periphery tightly abuts the top plate to further facilitate a substantially sealed arrangement.

Embodiments are also provided directed to the size and shape of the inserts and elongated tubes. Preferably the inserts received within the channels mounted within the tube manifold have a length that allows for a dampening of pressure from the tube manifold while still allowing for enough pressure to continue an air flow through the assembly to the reagent waste destination. The inserts received within the channels mounted within the tube manifold have a diameter roughly equal with the diameter of the synthesis wells but smaller than the diameter of the elongated tubes, which thus allows for the higher pressure of the synthesis wells to be reduced (i.e., dampened) within the elongated tube. In another embodiment, the elongated tubes have a length ranging between ½ to 5 inches, and in a further embodiment the tubes are between 3-4 inches, and in a further embodiment the tube is about 3.5 inches long. Preferably, the elongated tubes have a diameter ranging between 2-7 mm, and in a further embodiment the diameter is 5 mm. In a further embodiment, the ratio of the diameter of the opening of the elongated tube and the diameter of the opening of the insert or bottom of the well is between about 5:1 or 4:1.

According to one aspect, the present invention provides a method for reducing cross-contamination in synthesis reactions using a multi-well manifold assembly. The method of reducing cross-contamination when synthesizing in parallel a plurality of oligonucleotides in a plurality of synthesis wells, the method comprises putting each synthesis well in contact with a tube manifold to create a seal, wherein the manifold is in contact with an elongated tube to create a seal, said elongated tube having a diameter greater than a diameter of a manifold or synthesis well. In a further embodiment, the seals are created through the use of an o-ring. In a further embodiment a positive pressure differential is used to move reagent and air through the synthesis well, the pressure provided by an air pump or compressor. In one embodiment the pressure during synthesis is about 2 psi. For the deprotection step it is about 100 psi.

In another embodiment, the method of reducing cross-contamination comprises the steps of: a) inserting a plurality of elongated tubes within a full ring velocity stack plate, the full ring velocity stack plate comprising a full ring opening with a rim and a plurality of apertures for receiving the elongated tubes; b) inserting a tube manifold within the opening of the velocity stack plate, the tube manifold having a main body and top periphery with a top surface, wherein a plurality of channels extend through the top surface, top periphery and traverse through the main body of the tube manifold; c) inserting a plurality of inserts within the channels mounted within the tube manifold and aligning the inserts with the elongated tubes of the full ring velocity stack plate; and d) securing the tube manifold within the opening of the full ring velocity stack plate, wherein at least one sealing means if provided to substantially seal the tube manifold against the full ring velocity stack plate. The tube manifold is inserted and secured within the full ring opening of the full ring velocity stack plate and tightly abuts the sealing means and the stack plate to substantially form a seal between the tube manifold and the stack plate to reduce cross-contamination of continuous synthesis reactions during oligonucleotide synthesis.

### BRIEF DESCRIPTION OF THE DRAWING

The invention will be more fully understood and further advantages will become apparent when reference is had to the following detailed description of the preferred embodiments of the invention and the accompanying drawings, in which:

FIG. 1 is a schematic view of the multi-well manifold assembly in accordance with one embodiment of the present invention;

FIG. 2 is a sectional view of an embodiment of the multi-well manifold assembly with a synthesis plate placed on the top of the assembly;

FIG. 3 is a top view of an embodiment of the multi-well manifold assembly with a synthesis plate placed on the top of the assembly and a lid placed over the system for use in a reaction vessel;

FIG. 4 illustrates a cross-sectional view of the embodiment of FIG. 3;

FIG. 5 shows the results of synthesis in which oligonucleotide synthesis reactions were carried out in parallel in a multi-well apparatus, wherein Tables 1-2 in the figure illustrate cross-contamination using regular gas phase; and

FIG. 6 shows the results comparative of those in FIG. 5 of an experiment in accordance with the present invention in which oligonucleotide synthesis reactions were carried out in parallel in a multiwell synthesizer utilizing the multiwell assembly unit of the current invention, wherein Tables 3-5 in the figure illustrate reduced cross-contamination using the assembly system and method of the subject invention.

### DETAILED DESCRIPTION OF THE PREFERRED EMBODIMENTS

The flow through vessel or multi-well manifold assembly system and method of the subject invention is designed so that each individual well in the synthesis plate is not only isolated from the others and isolated from the bottom of the plate, but the pressure within the synthesis wells is better regulated to avoid the circulation of air over the wells when the air flow is disrupted, thus avoiding residual condensate contamination.

The present invention is directed to a system and method for reducing cross-contamination in synthesis reactions in multi-well synthesizers. The present invention substantially eliminates cross-contamination in synthesis reactions, saving time and money on further purification steps. More specifically, the present invention relates to a specific construction of a multi-well manifold assembly for use in multi-well synthesis and use of positive pressure that reduces cross-contamination between wells. Reduction of cross-contamination improves the efficiency and reproducibility of the synthesis reaction, such as, for example, oligonucleotide synthesis.

In one embodiment the multi-well manifold assembly for the reduction of cross-contamination in oligonucleotide synthesis comprises a full ring velocity stack plate having a full ring opening with a rim and a plurality of apertures for receiving a plurality of elongated tubes. The assembly further includes a tube manifold received within the full ring opening of the full ring velocity stack plate. The tube manifold is constructed having a main body and top periphery with a top surface. A plurality of channels extends through the top surface and top periphery, and traverse through the main body of the tube manifold. Pluralities of inserts are received within the channels mounted within the tube manifold, the inserts aligning with and mating with the elongated tubes. At least one sealing means is provided for sealingly engaging the stack plate, tube manifold, inserts and elongated tubes. As constructed, the tube manifold is inserted and secured within the full ring opening of the full ring velocity stack plate. It tightly abuts the sealing means and the stack plate to substantially form a seal between the tube manifold and the stack plate to reduce cross-contamination of continuous synthesis reactions in microfluidic devices.

In another contemplated embodiment the sealing means between the velocity stack plate and the tube manifold is a velocity stack o-ring located on the rim of the opening of the full ring velocity stack plate. Preferably the sealing means is an o-ring located on an underside of the top periphery of the tube manifold. Most preferably, the sealing means includes at least two o-rings. In this embodiment, at least one sealing means includes a velocity stack o-ring located on the rim of the opening of the full ring velocity stack plate and wherein at least one sealing includes an o-ring located on an underside of the top periphery of the tube manifold.

Other embodiments concern securement of the tube manifold from the full ring velocity stack plate by attachment means. Preferably, attachment means includes screwing of the tube manifold to the full ring velocity stack plate. Although, a number of known attachment means, such as clamping, locking or tacking may be utilized without departing from the scope of the invention. The assembly may include a plurality of assay plates for use with the system, selling the assembly as a kit; alternatively, the assay plates may be sold separately.

In one embodiment, the top periphery of the tube manifold extends outwardly from the main body forming a shelf. Upon insertion within the opening of the full ring velocity stack plate the shelf or top periphery tightly abuts the top plate to further facilitate a substantially sealed arrangement.

Embodiments are also provided directed to the size and shape of the inserts and elongated tubes. Preferably the inserts received within the channels mounted within the tube manifold have a length ranging between 5-30 mm. In another embodiment, the elongated tubes have a length ranging between 2-5 inches. Preferably, the elongated tubes have a length ranging between 3-4 inches.

According to one aspect, the present invention provides a method for reducing cross-contamination in continuous flow reactions using a multi-well manifold assembly. The method comprises the steps of: a) inserting a plurality of elongated tubes within a full ring velocity stack plate, the full ring velocity stack plate comprising a full ring opening with a rim and a plurality of apertures for receiving the elongated tubes; b) inserting a tube manifold within the opening of the velocity stack plate, the tube manifold having a main body and top periphery with a top surface, wherein a plurality of channels extend through the top surface, top periphery and traverse through the main body of the tube manifold; c) inserting a plurality of inserts within the channels mounted within the tube manifold and aligning the inserts with the elongated tubes of the full ring velocity stack plate; and d) securing the tube manifold within the opening of the full ring velocity stack plate, wherein at least one sealing means if provided to substantially seal the tube manifold against the full ring velocity stack plate. The tube manifold is inserted and secured within the full ring opening of the full ring velocity stack plate and tightly abuts the sealing means and the stack plate to substantially form a seal between the tube manifold and the stack plate to reduce cross-contamination of continuous amplification reactions in microfluidic devices.

The present invention has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited. This reduction of cross-contamination improves the efficiency, reproducibility and purity of the synthesis reaction, such as, for example, oligonucleotide synthesis.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, linking chemistry and amidite chemistry. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Stryer, L. (1995) Biochemistry (4th Ed.) Freeman, N.Y., Gait, Oligonucleotide Synthesis: A Practical Approach, 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

FIG. 1 shows a schematic view of an embodiment of the multi-well manifold assembly, shown generally at 10. The multi-well manifold assembly 10 includes a full ring velocity stack plate 11 having an opening 12 with a rim having a velocity plate o-ring 13. The o-ring 13 of stack plate 11 substantially traverses the entire perimeter of opening 12. A tube manifold 20 is received within opening 12 of stack plate 11. Tube manifold 20 is constructed having a main body 21 and top periphery 22 with a top surface 23. Top periphery 22 preferably extends outwardly from main body 21 forming a shelf that, upon insertion within opening 12 of stack plate 11 tightly abuts top plate o-ring 13 substantially forming a seal against the rim of opening 12 of the stack plate 11.

A plurality of channels 24 extend through the top surface 23, top periphery 22 and traverse through main body 21 of tube manifold 20. Inserts 25 are received within channels 24 and are mounted within tube manifold 20. Bottom tubes 26 mate with inserts 25, and extend below stack plate 11. An o-ring 30 is located between tube manifold 20 and stack plate 11. Tube manifold 20 is secured within top plate, preferably by screws 27 inserted through top periphery 22 through stack plate 11. The assembled multi-well apparatus 10 is coupled to the reaction device with vacuum and pressure controlled via bleed valve 40. Although the multi-well apparatus is shown having 96 wells, the multi-well manifold assembly 10 may include any number of wells. Top manifold assembly 10 is appointed to receive a multi-well assay plate 261 as illustrated via cross-sectional view in FIG. 2.

It has been found that at least one seal is needed at the bottom of the plate, which is believed to prevent cross-contamination of condensation contamination from neighboring assay wells. Though an o-ring seal is shown in FIG. 1, via seal at the bottom of the plate at the nozzle, a number of seals can be utilized without departing from the scope of the subject invention. Preferably, two seals are placed between the tube and the structure it mounts, one seals against the tube and one seals against the plate—as shown in FIG. 1 via o-rings 13, 30.

FIG. 2 shows a sectional view of an embodiment of the multi-well manifold assembly with a multi-well assay plate applied to the top of the assembly, shown generally at 200. Multi-well manifold assembly 210 is shown in the assembled condition with full ring velocity stack plate 211 with tube manifold 220 inserted within opening 212 of stack plate 211. A substantial seal results between the two components facilitated by the o-rings as shown in FIG. 1. Tube manifold 220 is constructed having a main body (not shown as is housed within velocity stack plate 211) and top periphery 222 with a top surface 223. Top periphery 222 preferably extends forms a shelf that tightly abuts stack plate 211. A plurality of channels 224 extend through the top surface 223, top periphery 222 and traverse through the tube manifold 220.

Inserts 225 are received within channels 224 and are mounted within tube manifold 220. Bottom tubes (i.e., elongated tubes) 226 mate with inserts 225, and extend below stack plate and are received in corresponding channels 251 in mixing chamber vessel 250 that can stand alone or be housed within a multiwall synthesizer instrument such as a MerMade™ DNA/RNA Synthesizer, a Dr. Oligo™ 96 or 192 synthesizer or other known multiwall synthesizers known in the art. Top manifold assembly 210 is shown receiving a multi-well assay plate 260 having multiple wells 261 housing assay wells 262. Multiwell synthesis plates are commercially available (see for example, Corning).

Top plate or full ring velocity stack plate 211 represents the processing plate, engaging tubes 226, o-ring seals (see FIG. 1), and mixing chamber vessel 250. The o-ring construction (see FIG. 1) has been found to optimize the seal between the top plate or full ring velocity stack plate 211 and the tube manifold 220 and mixing chamber vessel 250 while providing the ability to insert the top plate or full ring velocity stack plate 211 manually.

Tubes 226 are constructed as long tubes inserted in the full ring velocity stack plate 211, separating the flow of the assay samples and gas flow as the assembly is in the reaction vessel. Tubes 226 preferably have a larger cross-section area to allow the air flow to slow down and lose energy, reaching equilibrium, at a lower velocity. During oligonucleotide synthesis or deprotection steps, the pressure is disrupted when the pressure is reduced, such as when a vacuum is turned off or the liquid drains from some of the wells in the plate. Under conventional conditions, the pressure disruption leads to a brief bump of back-up pressure or causes a disruption in the air within and around the individual wells. In the present invention, the lower velocity in the tubes dampens that effect by absorbing the pressure disruption, thereby decreasing cross-contamination between the assay wells. The tubes also lesson condensation and condensation cross-over between the samples and/or the bottom of the mixing chamber vessel 250.

The bottom of the synthesis wells 262 are placed into contact with the inserts 225. Note that a gasket or o-ring can be used to form a seal between the synthesis well and the insert. Each insert 225 is in contact with the elongated tubes 226, and again there may be a gasket or o-ring that is used to ensure a seal between the insert and elongated tube.

FIG. 3 shows a top view of an embodiment of the multi-well manifold assembly with a multi-well assay plate applied to the top of the assembly and a lid placed over the system, shown generally at 300. FIG. 4 illustrates a cross-sectional view of the embodiment of FIG. 3 taken along A-A.

Referring to FIGS. 3 and 4, multi-well manifold assembly's 310 tube manifold 320 is sealingly secured via securing mechanism means, preferably screws, within full ring velocity stack plate 311. Inserts 325 of the tube manifold 320 are received within channels 324 and are received in long tubes 326. A multi-well assay plate/synthesis plate 360 is received on top of tube manifold 320. Assay wells 362 of multi-well assay plate 360 are received within the corresponding inserts 325 and tube 326 to which each are aligned. A lid 370 is engaged over the assay plate 360, pressing the plate 360 downward. In turn, inserts 325 are compressed by synthesis plate 360 and assay wells 362. Tube compression of the inserts 325 facilitates sealing between each separate well in the assay plate to facilitate prevention of cross-contamination. Top plate or full ring velocity stack plate 311 engages tubes 326, o-ring seals (see FIG. 1), and the mixing chamber vessel.

### Example 1

Sample experiments were conducted to demonstrate the reduction of cross-contamination in oligonucleotide synthesis reactions in a multi-well synthesizer using the method and system in accordance with one aspect of the present invention. Specifically the invention was utilized during the deprotection steps of synthesis. Contamination testing of one such experiment is shown in FIGS. 5 and 6, containing Tables 1-5. Specifically, as to FIG. 5, the tables show regular gas phase testing: Table 1 shows contamination testing wherein the samples were carried out via regular gas phase; Table 2 shows contamination testing wherein the samples were carried out via Regular gas phase wherein the membranes dried overnight. As to FIG. 6, the tables show gas phase testing using the system and method of the subject invention: Table 3 shows contamination testing wherein the samples were carried out via the subject inventions flow through gas phase—dried on vessel; Table 4 shows contamination testing wherein the samples were carried out via flow through gas phase—dried overnight #1; and Table 5 shows contamination testing wherein the samples were carried out via flow through gas phase—dried overnight #2.

Three plates were synthesized with 8 FAM-labeled, 15-mer oligonucleotides in column 6, while all other wells were empty. Oligonucleotide Sequence: /56-FAM/CTG AAG GGC GGT GAC was used. Membranes were transferred to a clean synthesis plate following synthesis. Plates were dried: 1 plate was dried using a 6 minute air cycle on the multiwell manifold assembly; 2 were allowed to sit for 24 hours, allowing liquid to evaporate. Ammonia gas was flowed through each plate for 90 minutes using the multiwell manifold assembly. Air was flowed through the plate for 6 minutes using the multiwell manifold assembly. Membranes and frits were removed from the column 6 of the synthesis plate and replaced with clean frits. The plate was eluted on an X-Cleaver using 3 mM base in ammonium hydroxide solution. Samples were dried on Zymark TurboVap 96 and re-eluted in 200 μL of IDTE 7.5 buffer using a BioTek μfill. 100 μL was sampled using a Perkin-Elmer MultiProbe II. Solution from column 6 was sampled. Fluorescence readings were taken on Spectramax Gemini XPS using an excitation wavelength of 484 and an emission wavelength of 525. The results are comparatively shown in FIGS. 5 versus 6. Note that RFU readings on wells relatively distant from column 6 (where synthesis actually occurred) are higher in plates in FIG. 5 where the synthesis and deprotection are performed by conventional means compared to FIG. 6 RFU readings.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Having thus described the invention in rather full detail, it will be understood that such detail need not be strictly adhered to, but that additional changes and modifications may suggest themselves to one skilled in the art, all falling within the scope of the invention as defined by the subjoined claims.

What is claimed is:

1. A method for reducing cross-contamination when synthesizing in parallel a plurality of oligonucleotides in a plurality of synthesis wells, said method comprising: putting each synthesis well in contact with a tube manifold to create a seal, wherein the tube manifold is in contact with an elongated tube to create a seal, said elongated tube having a diameter greater than a diameter of a tube manifold or synthesis well.

2. The method of claim 1 wherein the seals are created through the use of an o-ring.

3. The method of claim 1 wherein a positive pressure differential is used to move reagent and air through the synthesis well.

4. An apparatus for reducing cross-contamination in multiwell parallel oligonucleotide synthesis, said apparatus comprising:
a. a multiwell synthesis plate, said multiwell synthesis plate containing multiple synthesis wells wherein each synthesis well has a bottom diameter;
b. a tube manifold, said tube manifold comprising multiple wells wherein each well has an insert of a diameter equal or greater than the bottom diameter of the synthesis well, and wherein the insert provides a seal when in contact with the synthesis well;
c. an elongated tube having a diameter greater than the synthesis well bottom diameter and wherein when in contact with the insert a seal is made; and
d. an air supply to provide a positive pressure differential.

5. The apparatus of claim 4 further comprising a gasket or o-ring between the synthesis well and insert.

6. The apparatus of claim 4 further comprising a gasket or o-ring between the insert and the elongated tube.

7. A multi-well manifold assembly for the reduction of cross-contamination in continuous flow reactions, comprising:
a. a full ring velocity stack plate having a full ring opening with a rim and a plurality of apertures for receiving a plurality of elongated tubes;
b. a tube manifold received within the full ring opening of the full ring velocity stack plate;
c. the tube manifold having a main body and top periphery with a top surface, wherein a plurality of channels extend through the top surface, top periphery and traverse through the main body of the tube manifold;
d. a plurality of inserts received within the channels mounted within the tube manifold, the inserts aligning with and mating with the elongated tubes;
e. at least one sealing means;
wherein the tube manifold is inserted and secured within the full ring opening of the full ring velocity stack plate and tightly abuts the sealing means and the stack plate to substantially form a seal between the tube manifold and the stack plate to reduce cross-contamination of continuous synthesis reactions in microfluidic devices.

8. A multi-well manifold assembly as recited by claim 7, wherein the inserts are gas phase PVC EPDM inserts.

9. A multi-well manifold assembly as recited by claim 7, wherein the sealing means is a velocity stack o-ring located on the rim of the opening of the full ring velocity stack plate.

10. A multi-well manifold assembly as recited by claim 7, wherein the sealing means is an o-ring located on an underside of the top periphery of the tube manifold.

11. A multi-well manifold assembly as recited by claim 7, wherein the sealing means includes at least two o-rings.

12. A multi-well manifold assembly as recited by claim 11, wherein at least one sealing means includes a velocity stack o-ring located on the rim of the opening of the full ring velocity stack plate and wherein at least one sealing includes an o-ring located on an underside of the top periphery of the tube manifold.

13. A multi-well manifold assembly as recited by claim 7, wherein the tube manifold is appointed to be removably attached to the full ring velocity stack plate by attachment means.

14. A multi-well manifold assembly as recited by claim 7 comprising a multi-well assay plate.

15. A multi-well manifold assembly as recited by claim 7, wherein the top periphery of the tube manifold extends outwardly from the main body forming a shelf that, upon insertion within the opening of the full ring velocity stack plate tightly abuts the top plate.

16. A multi-well manifold assembly as recited by claim 7, wherein the inserts received within the channels mounted within the tube manifold have a length ranging between 5-30 mm.

17. A multi-well manifold assembly as recited by claim 7, wherein the inserts received within the channels mounted within the tube manifold have a diameter ranging between 1-5 mm.

18. A multi-well manifold assembly as recited by claim 7, wherein the elongated tubes have a length ranging between 2-5 inches.

19. A multi-well manifold assembly as recited by claim 7, wherein the elongated tubes have a diameter ranging between 4-8 mm.

20. A method for reducing cross-contamination in continuous flow reactions using a multi-well manifold assembly, comprising the steps of:
a. inserting a plurality of elongated tubes within a full ring velocity stack plate, the full ring velocity stack plate comprising a full ring opening with a rim and a plurality of apertures for receiving the elongated tubes;
b. inserting a tube manifold within the opening of the velocity stack plate, the tube manifold having a main body and top periphery with a top surface, wherein a plurality of channels extend through the top surface, top periphery and traverse through the main body of the tube manifold;
c. inserting a plurality of inserts within the channels mounted within the tube manifold and aligning the inserts with the elongated tubes of the full ring velocity stack plate;
d. securing the tube manifold within the opening of the full ring velocity stack plate, wherein at least one sealing means if provided to substantially seal the tube manifold against the full ring velocity stack plate.
wherein the tube manifold is inserted and secured within the full ring opening of the full ring velocity stack plate and tightly abuts the sealing means and the stack plate to substantially form a seal between the tube manifold and the stack plate to reduce cross-contamination of continuous synthesis reactions in microfluidic devices.
